(19) United States
(12) Patent Application Publication
(10) Pub. No.: US 2014/0163379 A1
(43) Pub. Date: Jun. 12, 2014

(54) METHOD AND SYSTEM FOR DETECTING AND ASSESSING BRAIN INJURIES USING VARIABILITY ANALYSIS OF CEREBRAL BLOOD FLOW VELOCITY

(71) Applicant: Vladislav Bukhman, East Northport, NY (US)

(72) Inventor: Vladislav Bukhman, East Northport, NY (US)

(21) Appl. No.: 13/709,602

(22) Filed: Dec. 10, 2012

(51) Int. Cl. A61B 8/06

(52) U.S. Cl. CPC A61B 8/06
USPC 600/454

(57) ABSTRACT

Disclosed herein are method and system for detecting and assessing brain injuries and conditions. The medical system is capable of detecting and assessing the severity of TBI is present. The determination is performed by multiscale analysis of complexity of cerebral blood flow velocity oscillations.

### CROSS REFERENCE TO RELATED APPLICATIONS

This application claims the benefit of U.S. Provisional Application No. 61/608,857 filed on Mar. 9, 2012 the disclosure of which is incorporated by reference as if fully set forth herein.

### FIELD OF THE INVENTION

This invention relates generally to medical device systems and, more particularly, to medical device systems and methods capable of detecting and quantitative assessing of brain injuries and other conditions in particular traumatic brain injuries (TBI).

### BACKGROUND

TBI contributes significantly to military combat trauma and civilian morbidity and mortality. The Department of Defense trauma registry for data on all care rendered to military trauma care system indicates that 8% of all war-related wounds occurred to the head. TBI accounts for over one-quarter of all injury hospitalizations during Operation Iraqi Freedom and Operating Enduring Freedom. Prevention of secondary brain injury is the goal of neurosurgical, critical care and neurological management strategies in TBI population with emphasis on control of cerebral ischemia and ICP. Unfortunately, many patients with moderate and severe TBI do not survive the acute phase of their injuries and those who do, including patients with mild TBI, may often suffer long term cognitive and physical disabilities. Patients with mild TBI also often will be misdiagnosed and not treated properly. The current state of neurologic monitoring and prognostication in TBI patients lacks accuracy in assessing the current condition of a patient and determining which patients survive and what their long term deficits and outcomes will be.

Transcranial Doppler (TCD) is an ultrasound technique primarily used to measure cerebral blood flow velocity (CBFv) in cm/sec. There is numerous evidence that TCD serves as a complement to clinical assessment in acute neurologic conditions. Studies of TCD in civilian TBI have suggested utility in this setting TCD has proven useful as a prognostic tool in patients with mild to moderate and severe TBI and could predict those at risk for secondary neurologic deterioration (edema, herniation, and hydrocephalus) within the first hours or one week after TBI. However, today TCD clinical utilization in TBI population is limited to diagnostic evaluation of posttraumatic cerebral vasospasm and abnormally high intracranial pressure (ICP) but imperfect TCD information is extracted for prognostication. For example, fewer studies have assessed cerebral blood flow oscillations in particular CBFv moment-to-moment variability, and its potential diagnostic and prognostic potential in TBI patients. In a study of CBFv variability via TCD in a cohort of TBI patients it was suggested that sustained CBFv variability, despite a reduction in arterial pressure variability, was a potential mechanism for the protection of cerebral tissue oxygenation (Turalska M, Latka M, Czosnyka M, Pierzchala K, West B J. Generation of very low frequency cerebral blood flow fluctuations in humans. Acta Neurochir Suppl. 2008; 102:43-7).

A loss of variability portends increased morbidity and mortality as has been extensively investigated in cardiology: a reduction in heart rate variability is associated with poorer prognosis and/or increased mortality risk in patients with coronary artery disease, dilated cardiomyopathy, congestive heart failure, and post cardiac infarction patients.

The cerebral circulation shows both structural and functional complexity. However, it is well known that high organized biological systems, such as the brain, operate as a chaotic system which is far from a stable equilibrium and could be characterized by unpredictability (Panerai R. Complexity of the human cerebral circulation. Phil Trans R Soc, 2009; 367:1319-1336). Unpredictability in high organized biological systems is referred as complexity.

The present invention represents a method of objective assessment of TBI based on the analysis of the CBFv variability that evaluates complexity of the dynamics of CBFv and calculates Neurovascular Complexity Index (NCI). The method is based on the analysis of the dynamics of CBFv using chaos theory. Chaos is common in nature. Many natural phenomena can also be characterized as being chaotic, such as the weather, living organism systems, electronic circuits, or chemical reactions. A chaotic system is characterized by unpredictability, which simply means that one cannot predict how a system will behave in the future, on the basis of a series of observations over time. The evaluation of complexity of cerebral circulation by measuring the variability of CBFv provides information on whether the neurovascular system, which facilitates the autoregulation, is impaired or intact. If complexity is low, then the neural system is impaired and the autoregulation may not be adequate.

### SUMMARY OF THE INVENTION

In one aspect of the present invention, a method of assessing TBI in a patient is provided. In one embodiment, the method comprises receiving time series data relating to a cerebral blood flow velocity (CBFv) of the patient's cerebral arteries, e.g. middle cerebral artery (MCA); measuring Neurovascular Complexity Index (NCI) using multiscale complexity analysis of CBFv time series of the patient; comparing the determined NCI(t) to a threshold NCI(c) related to NCI value of persons without history of TBI and other neurological or psychological disorders; and providing an output indicative of TBI or other conditions and/or an output of indicative severity of TBI or other conditions. The present invention uses TCD as the devise and CBFv as the input for applying multiscale complexity analysis. However, other devices and cerebral blood flow parameters can be used for detecting and assessing TBI and other conditions by applying multiscale complexity analysis.

### BRIEF DESCRIPTION OF THE DRAWINGS

The invention may be understood by reference to the following description taken in conjunction with the accompanying drawings, in which like reference numerals identify like elements, and in which:

FIG. 1 shows plotted CBFv time series recorded by TCD device.

FIG. 2 illustrates a tale distribution graph for a normal patient without history of TBI and other neurological conditions.

FIG. 3 shows NCI value for the normal patient of FIG. 2.

FIG. 4 illustrates tale distribution graphs for patients with TBI of different categories of severity and a tale probability distribution graphs of control group without history of TBI;

FIG. 5 shows a graph of NCI values for the same control group and patients with TBI as on FIG. 3;

FIG. 6 is a Box-and-Whisker plot of NCI values for the control group and patients with TBI;

FIG. 7 is a Box-and-Whisker plot of NCI values for sport related mild TBI (concussions);

FIG. 8 illustrates follow up NCI values for a patient with suicidal west IED blast brain injury.

FIG. 9 shows a patient with stroke after severe blast brain injury.

FIG. 10 illustrates monitoring of a patent with subarachnoid hemorrhage (SAH) and hydrocephalus in induced coma state on days 6 and 7.

FIG. 11 demonstrates recovering progress of a patient with sever TBI.

FIG. 12 shows a worsening condition of a patent with sever TBI.

FIG. 13 shows a NCI score on day 1 and day 3 of a patient with severe TBI.

The patent was dead at day 11.

FIG. 14 is a stylized diagram of a medical system that includes an ultrasound TCD unit and a digital signal processing (DSP) unit, in accordance with one illustrative embodiment of the present invention;

FIG. 15 is a functional block diagram of medical system, in accordance with one illustrative embodiment of the present invention;

While the invention is susceptible to various modifications and alternative forms, specific embodiments thereof have been shown by way of example in the drawings and are herein described in detail. It should be understood, however, that the description herein of specific embodiments is not intended to limit the invention to the particular forms disclosed, but on the contrary, the intention is to cover all modifications, equivalents, and alternatives falling within the spirit and scope of the invention as defined by the appended claims.

### DETAILED DESCRIPTION OF SPECIFIC EMBODIMENTS

Illustrative embodiments of the invention are described herein. In the interest of clarity, not all features of an actual implementation are described in this specification. In the development of any such actual embodiment, numerous implementation-specific decisions must be made to achieve the design-specific goals, which will vary from one implementation to another. It will be appreciated that such a development effort, while possibly complex and time-consuming, would nevertheless be a routine undertaking for persons of ordinary skill in the art having the benefit of this disclosure.

This document does not intend to distinguish between components that differ in name but not function. In the following discussion and in the claims, the terms “including” and “includes” are used in an open-ended fashion, and thus should be interpreted to mean “including, but not limited to.” The word “or” is used in the inclusive sense (i.e., “and/or”) unless a specific use to the contrary is explicitly stated.

In one embodiment, the present invention provides a method of diagnosing and assessing of TBI and other brain conditions.

The data relating to CBFv of the patient's brain can be gathered by any of a number of techniques. For example, data relating to CBFv may be gathered by a transcranial Doppler device, such as Doppler-Box TCD system offered by Compumedics DWL, Germany. In one embodiment, the data relating to the CBFv data time series may be related to the middle cerebral artery (MCA). Those skilled in the art having benefit of the present disclosure would appreciate that time series of CBFv data from other brain arteries (e.g., Anterior Cerebral, Posterior Cerebral, Internal Carotid, etc.) may be used and still remain within the spirit and scope of the present invention.

Recent researches provide new evidence that the human brain organizes itself to operate “on the edge of chaos”, at critical transition point between randomness and order (Kitzbichler et al. Broadband Criticality of Human Brain Network Synchronization. PLoS Computational Biology, Mar. 20, 2009). System on the edge of chaos are said to be in a state of Self-Organized Criticality (SOC). These systems are on the boundary between stable orderly behavior and unpredictable world of chaos. SOC emerges from studies of complex systems of interactive elements. SOC is one of important discoveries made over the latter half of the 20th century relating to complexity in nature.

Takens' theorem states that it is possible to reconstruct of a high dimensional system by observing a single output variable (F. Takens (1981). “Detecting strange attractors in turbulence”. In D. A. Rand and L.-S. Young. Dynamical Systems and Turbulence, Lecture Notes in Mathematics, vol. 898. Springer-Verlag. pp. 366-381).

The proposed method utilizes Cerebral Blood Flow velocity (CBFv) data measured by Transcranial Doppler (TCD) as the single output variable however other devices and other parameters related to cerebral blood flow may be used. CBFv data is easy to obtain, very accurate, and not susceptible to noise, as compared to EEG, metabolic analysis, or other available technologies. Measurements are taken from MCA because it supplies nutrients to almost all of the brain tissue. Data is collected from the left MCA (LMCA) or right MCA (RMCA) or from both, and CBFv is expressed in centimeter per second (cm/s). The method and system comprises of Transcranial Doppler and a software measures the critical dynamics of CBFv and relates it to neurological and neuropsychiatric disorders in particular traumatic brain injuries.

A chaotic system is characterized by ‘unpredictability’, which simply means that one cannot predict how a system will behave in the future, on the basis of a series of observations over time. For complex biological systems ‘unpredictability’ is usually referred as complexity. In order to survive, the system should have a minimum amount of complexity. By analyzing the level complexity one may determine if the brain in a normal state or in an impaired state.

There are many methods for the evaluation of complexity of high-dimensional, SOC systems. The most popular is measuring the complexity of the system by using entropy. However, entropy-based or any other methods of measuring complexity at one scale may provide misleading results while assessing threshold levels of complexity because data with different properties may produce vastly different results.

The present invention introduces Multiscale Complexity Analysis (MSCA) of CBFv oscillations using a Complementary Probability Cumulative Distribution Function also called Tail Distribution, adapted for the analysis of CBFv oscillations. Tail Distribution is defined as

F̅(x)=P(X>x)

where P is the probability that the random variable X takes on a value more than x.

TCD outputs the time series of CBFv data as a set of measured velocities, {v₁,v₂,v₃, . . . vᵢ,vᵢ₊₁, . . . , vₙ}. CBFv time series is transform to the time series of differences of successive points {d₁,d₂,d₃, . . . dᵢ,dᵢ₊₁, . . . dₙ₋₁} where dᵢ is the absolute difference, |vᵢ₊₁−vᵢ| between successive points vᵢ₊₁ and vᵢ. All vᵢ and dᵢ values are in cm/s.

Pᵢ is the Probability in percentages that the absolute difference between the measured values of Successive Points is more than x.

Pᵢ=P(dᵢ>x), where dᵢ is |vᵢ₊₁−vᵢ|

Pᵢ value at value x=a is the measurement of complexity at scale a. Plotting Pᵢ values with x varying from 0 to maximum of dᵢ provides a graph of multiscale complexity of CBFv oscillations.

Neurovascular Complexity Index (NCI) is calculated as a Tale distribution Function (TDF) density which is defined as

$$TDF [ a \leq x \leq b ] = {\int }_{a}^{b} f ( x ) d x$$

For discrete values the equation becomes

$$TDF [ 0 \leq x \leq max ] = {\sum }_{i = 0}^{max} {p}_{i} ( {x}_{i} )$$

where max is an empirical cut off value representing maximal difference between the largest and smallest two consecutive values of vᵢ and vᵢ₊₁.

CBFv time series values of the signal recorded with high sampling rate are affected by heart beats. In the present invention the CBFv time series are re-samples at 1 Hz frequency to mitigate the effect of heart beats.

Although not so limited, a system capable of implementing embodiments of the present invention is described below.

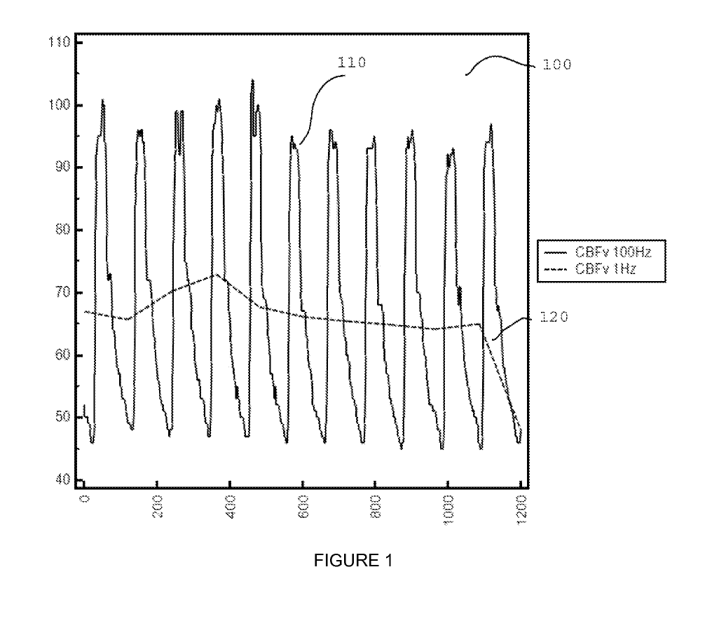

FIG. 1 is plotted CBFv graphs 100 for CBFv sampled at 100 Hz 110 and re-sampled at 1 HZ 120. It shows effect of the heart beats in 100 Hz line. The 1 Hz line shows that fluctuations related to heartbeats disappear.

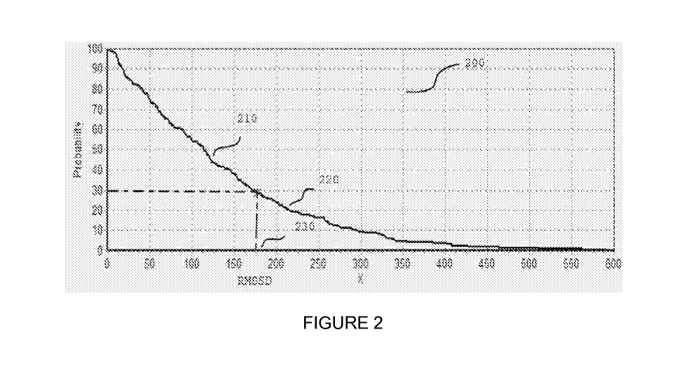

FIG. 2 demonstrates a median tale distribution graph 200 for a control group of patients without history of TBI and other neurological conditions. The graph shows that the tale distribution of CBFv variability of time series of successive differences {d₁,d₂,d₃, . . . dᵢ,dᵢ₊₁, . . . dₙ₋₁} follows a power law Pᵢ=k/x̂a and has a head tale 210 to long tale 220 ratio 70:30 relative to the Root Mean Square Successive Differences (RMSSD) 230 of the time series {d₁,d₂,d₃, . . . dᵢ,dᵢ₊₁, . . . dₙ₋₁}. A power law is imprinted in many complex biological complex systems including the brain. Deviations from the median power law tale distribution graph of variability of CBFv manifests abnormal state of the brain.

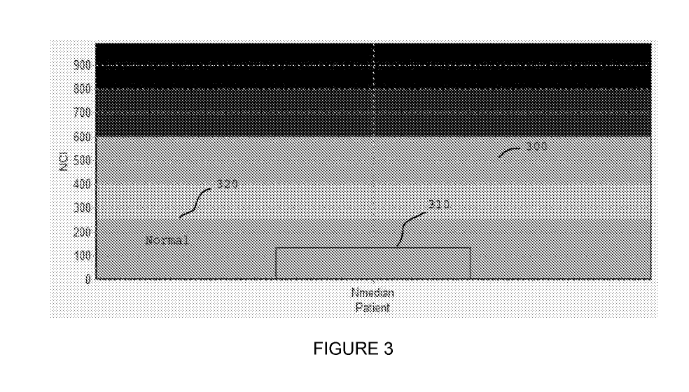

FIG. 3 is a graph 300 showing the NCI value 310 for the variability of CBFv of the same median patent as in FIG. 2. The normal threshold 320 is determined as 95% percentile of NCI values for the control group.

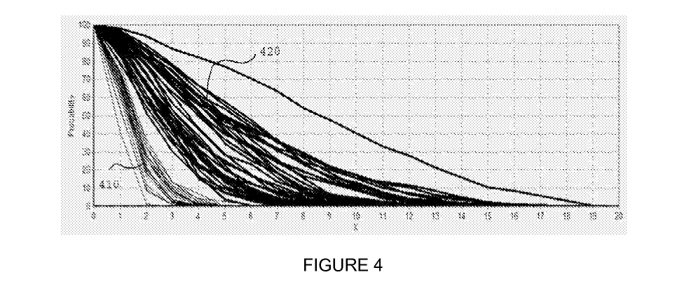

FIG. 4 depicts tale distribution curves for the control group 410 without history of TBI (tiny lines) and patients with TBI 420 (bold lines) of different categories of severity. It shows that all TBI patients have different tale distribution patterns than the control group. TCD measurements of TBI patients were performed in emergency room (ER) and severity of TBI were evaluated using Glasgow Coma Scale summary (ERGCSsum).

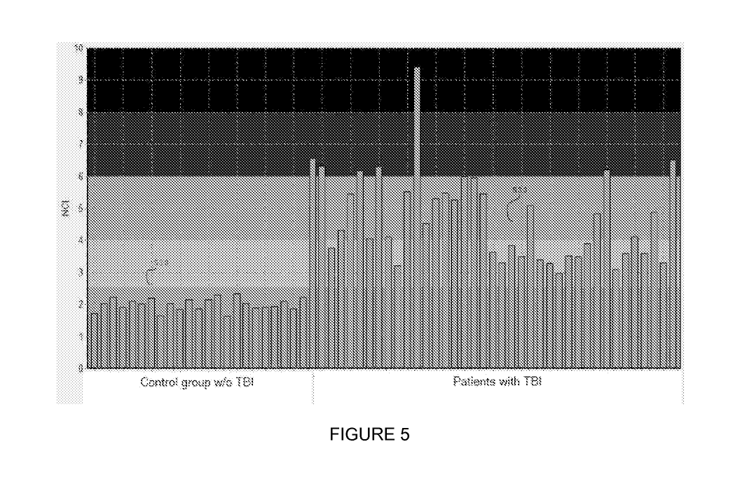

FIG. 5 shows a chart for the same as in FIG. 4 control 510 and TBI 520 groups with NCI values. It demonstrates how NCI values change related to severity of TBI. The more severe TBI has higher NCI value.

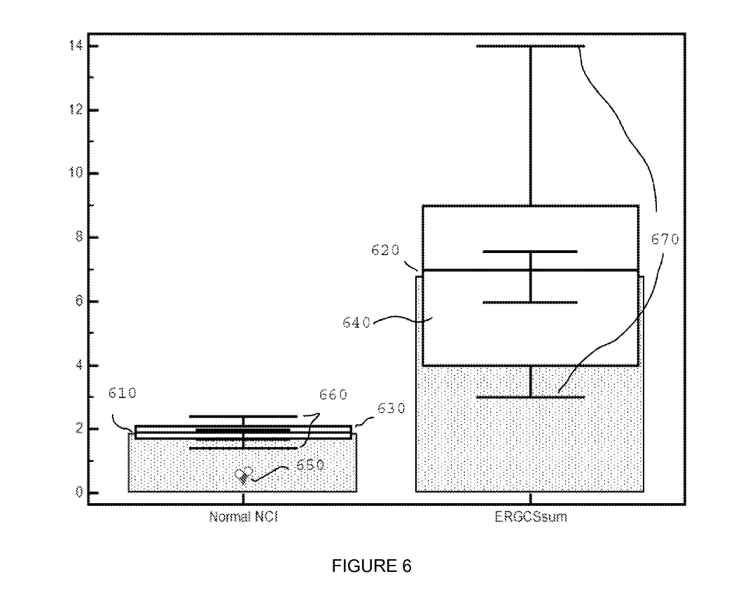

FIG. 6 is a chart of statistical analysis of NCI values for the same control and TBI groups as in FIG. 5. Box-and-Whisker plot shows mean lines 610 and 620, 75% percentile 630 and 640, outliers 650 and max/min values 660 and 670 for the same control and TBI groups as in FIG. 4.

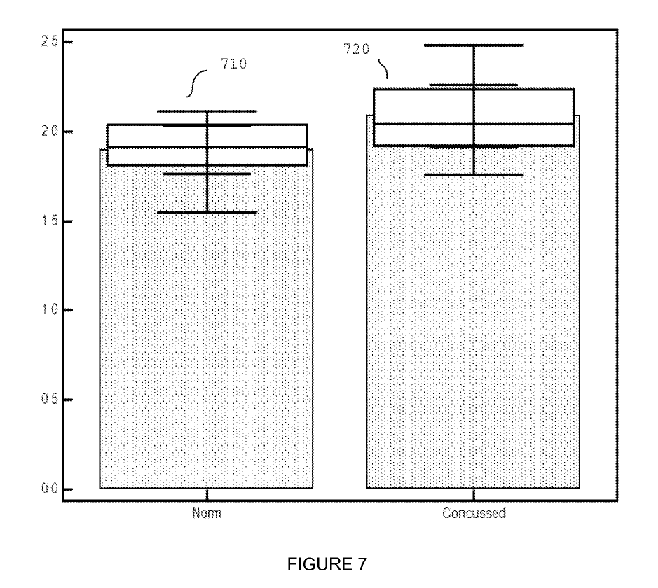

FIG. 7 illustrates the statistical difference using Box-and-Whisker plot for athletes without history of mTBI/concussion 710 and athletes with concussion 720 within 3 days of concussion events.

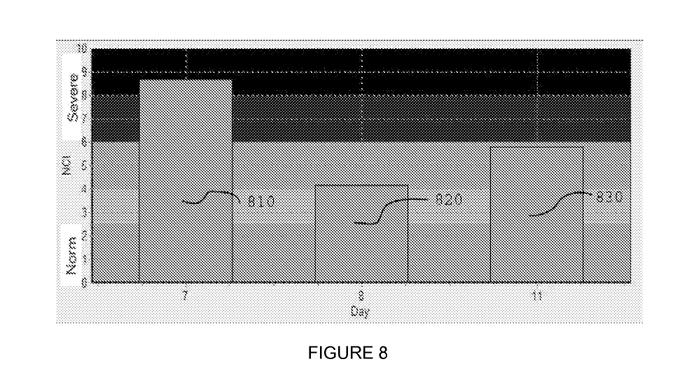

FIG. 8 shows NCI values for TCD measurements taken at day seven 810, eight 820 and eleven after suicidal IED blast injury with diagnosed vasospasm at day seven.

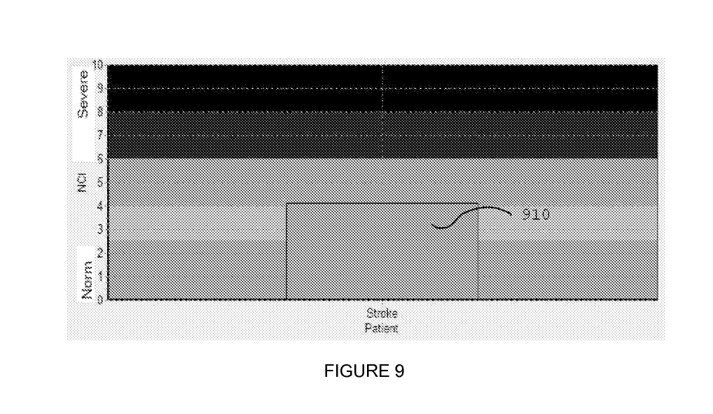

FIG. 9 shows NCI value above normal 910 for a patient with confirm acute ischemic stroke.

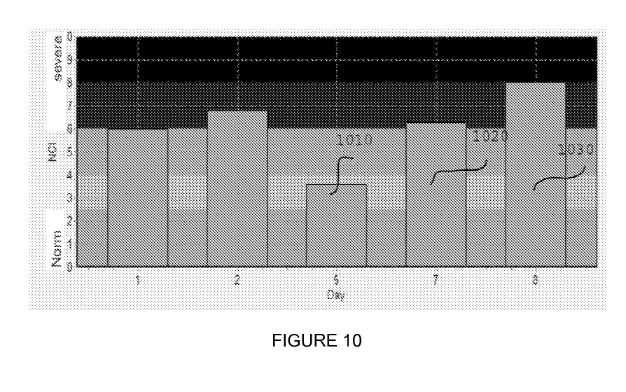

FIG. 10 shows NCI values of 6 days monitoring of a patient with subarachnoid hemorrhage and hydrocephalus. The condition got better after coiling of aneurysm on day five 1010 but getting worse on day seven 1020 with coma on day eight 1030.

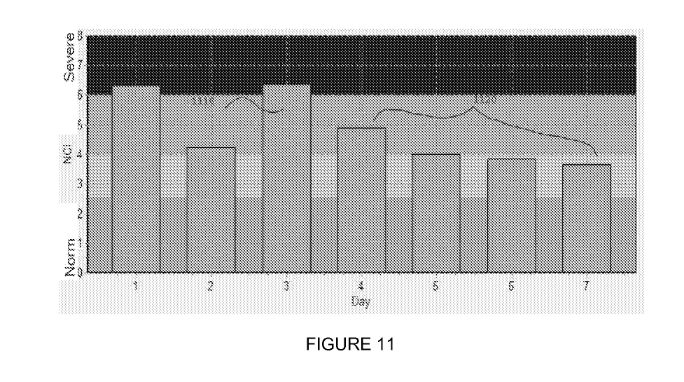

FIG. 11 demonstrates a good recovery progress during one week monitoring of a patient with severe TBI. The patient had a secondary injury which is result of complications after the primary injury on day three 1110 with gradual steady recovery on days 4, 5, 6 and seven 1120.

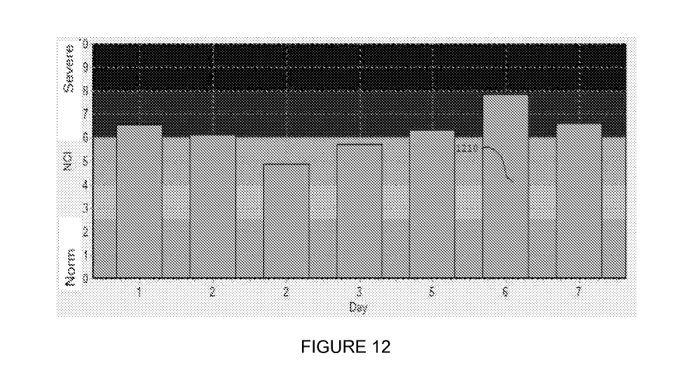

FIG. 12 shows an example of bad recovery with severe disability outcome. The condition was improved on day two and was getting worse on days 3-7 with NCI score sharply increased on day six 1210.

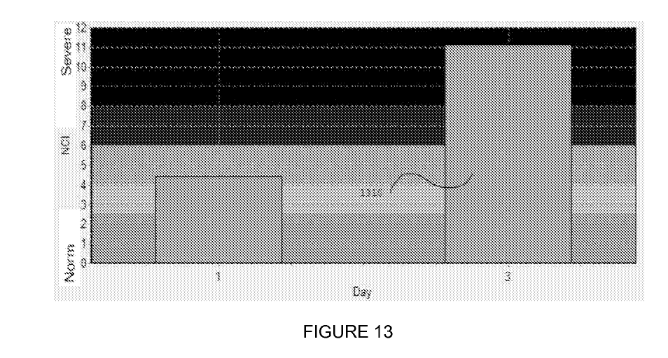

FIG. 13 shows NCI values for a patient with deadly outcome after severe TBI. The condition was suddenly significantly deteriorated of day three 1310 and the patient was pronounced dead at day 7.

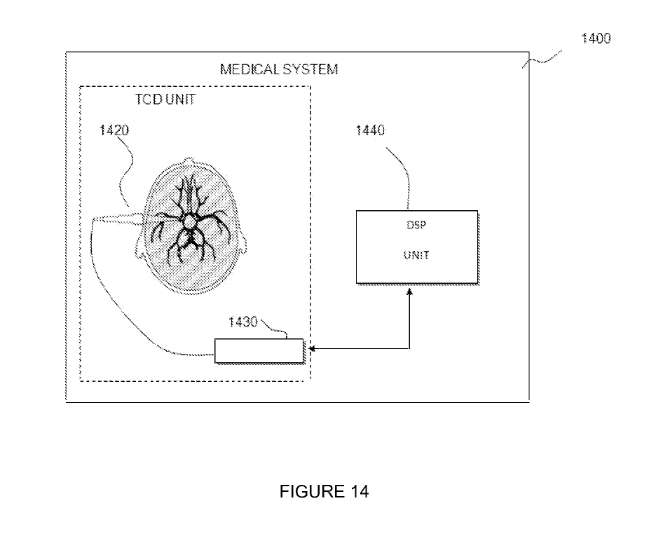

FIG. 14 depicts a stylized medical system 1400 for implementing one or more embodiments of the present invention. Sound waves emitted by a probe 1420 are transmitted through the relatively thin temporal bone and reflected from red blood cells moving in the basal arteries of the brain. The signal is processed, the velocity of the blood flow is determined and TBI is detected and assessed using multiscale complexity analysis in DSP unit 1430.

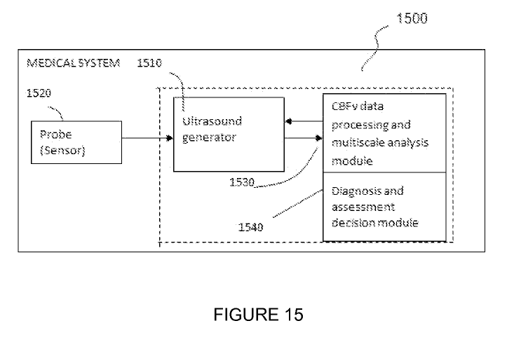

Turning now to FIG. 15, a functional block diagram depiction 1500 of a medical system 1500 is provided, in accordance with one illustrative embodiment of the present invention. The medical device 1500 may comprise a generator 1510 and the CBF data processing unit. The ultrasound probe 1520 emits a high-pitched sound wave from the ultrasound generator 1510 which then bounces off of various materials to be measured by the same probe. The speed of the blood in relation to the probe causes a phase shift, wherein the frequency is increased or decreased. The CBF data processing and analysis module 1530 measures the differences and calculates the velocity of the blood flow, CBFv. The CBFv data is analyzed using multiscale complexity analysis, a diagnosis is made and the severity of TBI is assessed in the diagnosis and assessment decision module 1540.

All of the methods and apparatuses disclosed and claimed herein may be made and executed without undue experimentation in light of the present disclosure. While the methods and apparatus of this invention have been described in terms of particular embodiments, it will be apparent to those skilled in the art that variations may be applied to the methods and apparatus and in the steps, or in the sequence of steps, of the method described herein without departing from the concept, spirit, and scope of the invention, as defined by the appended claims. It should be especially apparent that the principles of the invention may be applied to selected cerebral arteries other than, or in addition to, the MCA as well for other neurological disorders and conditions to achieve particular results.

The particular embodiments disclosed above are illustrative only as the invention may be modified and practiced in different but equivalent manners apparent to those skilled in the art having the benefit of the teachings herein. Furthermore, no limitations are intended to the details of construction or design herein shown other than as described in the claims below. It is, therefore, evident that the particular embodiments disclosed above may be altered or modified and all such variations are considered within the scope and spirit of the invention. Accordingly, the protection sought herein is as set forth in the claims below.

What is claimed:

1. A method of detecting and assessing a brain injury and condition in a patient, comprising: receiving data relating to cerebral blood flow velocity of patient's brain; determining at least one NCI parameter wherein NCI is a chaos theory complexity parameter associated with variability of cerebral blood flow velocity of patient's brain; comparing the at least one complexity parameter with thresholds; providing an output indicative of a brain injury, indicative of severity of the brain injury, or other conditions based on the comparison.

2. The method of claim 1, wherein is the at least one complexity parameter is the density of tale distribution.

3. The method of claim 2, wherein the tale distribution is the probability that the random variable X takes on a value more than x.

4. The method of claim 3, wherein the random variable X belongs to the time series of differences of successive points {d₁,d₂,d₃, . . . dᵢ,dᵢ₊₁, . . . dₙ₋₁} where dᵢ is the absolute difference, |vᵢ₊₁−vᵢ| between successive points vᵢ₊₁ and vᵢ of CBFv measurements.

5. The method of claim 4 wherein the x is variable from 0 to maximal value dᵢ from the time series {d₁,d₂,d₃, . . . dᵢ,dᵢ₊₁, . . . dₙ₋₁}.

6. A medical system for detecting and assessing TBI and conditions comprising: an ultrasound probe, a generator of ultrasound waves, CBF data processing unit.

7. The medical system of claim 6, wherein the generator emits a high-pitched sound wave through the ultrasound probe which then bounces off of various materials to be measured by the same probe.

8. The medical system of claim 6, wherein ultrasound waves are transmitted through thin temporal bone and reflected from red blood cells moving in the basal arteries of the brain.

9. The medical system of claim 6, wherein reflected waves are processed in the processing unit.

10. The medical system of claim 6, wherein, processing module has sound waves processing unit for calculation of CBFv and CBFv analysis unit.

11. The medical system of claim 6, wherein CBFv analysis unit perform multiscale complexity analysis of CBFv.

12. The medical system of claim 6, wherein processing unit perform TBI detection and assessment analysis using results of multiscale complexity analysis of CBFv.
